(19) United States
(12) Patent Application Publication
(10) Pub. No.: US 2004/0258634 A1
(43) Pub. Date: Dec. 23, 2004

(54) Compositon for oral hygiene comprising a fluoride ion vector and an antioxidant

(76) Inventors: Jean-Louis Cazor, Saint Antoine (FR); Francois Lhuisset, Gentilly (FR); Pascale Robineau, Palaiseau (FR)

Correspondence Address: Sanofi Synthelabo Inc, Patent Department, 9 Great Valley Parkway, PO Box 3026, Malvern, PA, 19355, US

(21) Appl. No.: 10/481,718

(22) PCT Filed: Jul. 3, 2002
(86) PCT No.: PCT/FR02/02313

(30) Foreign Application Priority Data
Jul. 4, 2001 (FR) 01/08852

(51) Int. Cl. A61K 7/18

(52) U.S. Cl. 424/52

(57) ABSTRACT

The invention relates to a composition for oral hygiene that is suitable for diabetics.

The composition according to the invention comprises at least one fluoride-ion vector and at least one antioxidant, and may be especially in the form of a dentifrice, a chewing gum, a mouthwash or a spray.

The invention also relates to the use of at least one fluoride-ion vector and of at least one antioxidant for the preparation of a composition for oral hygiene to prevent and/or combat oral pathologies in diabetics.

The invention relates to compositions for oral hygiene and to uses thereof.

Oral pathologies are common in diabetics. Furthermore, in the case of these individuals, an infection of the oral environment can cause an imbalance in the diabetes and result in dissemination of the microorganisms throughout the body. Several studies have thus shown that an improvement in the condition of the gums might facilitate the control of glycemia (Mealey, B. (2000) Diabetes and periodontal diseases, J. Periodontol. 71, 664-678).

It is also known that diabetes multiplies by a factor of about three the risk of developing a periodontal disease (Emrich L. C. et al (1991) Periodontal disease in non-insulin dependent diabetes mellitus, J. Periodontol. 62, 123-130). Other studies have shown that the frequency of caries and the prevalence of stomatitis of various origins are increased in diabetics (Twetman S. et al, (1992) Two-year longitudinal observations of salivary status and dental caries in children with insulino-dependent diabetes mellitus, Pediatr. Dent. 14, 184-188).

The relatively high frequency of the oral pathologies mentioned above in diabetics is especially linked with the following dysfunctions. Firstly, hyperglycemia causes nonenzymatic glycosylation and oxidation of lipids and proteins, and also the activation of RAGE (“Receptor for Advanced Glycation End products”) receptors, present on the membrane surface of many tissues. These biochemical reactions result especially in an increase in the oxidative stress of cells and, finally, the development of a proinflammatory environment, which is favorable to the formation of lesions and results in deficiency of the repair mechanisms.

Oxygen free radicals are in fact responsible for the impairment of DNA and of constitutive molecules such as collagen and elastin, the destruction of mucopolysaccharides, deleterious changes in the composition of membranes, and vascular lesions.

To combat the harmful effects of oxygen free radicals, it has been proposed to administer compounds with antioxidant activity to diabetics. It has thus been shown, for example, that an oral supplementation with an antioxidant such as vitamin E reduces the risks of retinopathy and nephropathy in diabetics, these complications being associated with diabetes-induced vascular dysfunction (Bursell S. E. et al, (1999) High Dose Vitamin E Supplementation Normalizes Retinal Blood Flow and Creatinine Clearance in Patients with type I Diabetes, Diabetes Care 22, 1245-1251).

The oral activity of vitamin E has, however, undergone little investigation. In vitro, it is capable of reducing the oxidative stress suffered by human oral epithelial cells in the presence of hydrogen peroxide (Royack G. A. et al, (2000) Response of human oral epithelial cells to oxidative damage and the effect of vitamin E, Oral Oncology 36, 37-41).

When applied locally to oral regions affected by mycosis—this complaint often being caused by Candida albicans and frequent in diabetics—vitamin E results in the rapid resolution of the lesions (Wadleigh R. G. et al, (1992) Vitamin E in the treatment of chemotherapy-induced mucositis, Am. J. Med. 92, 481-484).

The effects of vitamin E in topical application on the condition of the gums have moreover been described as nonexistent (Lange D. E. et al, (1975) Management of gingival inflammation with active ingredients in toothpaste, Dtsch. Zahnarztl. Z. 30, 382-384; Cohen R. E. et al, (1991) Effect of vitamin E gel, placebo gel and chlorhexidine on periodontal disease, Clin. Prev. Dent. 13, 20-24).

The frequency of oral pathologies in diabetics is associated, on the one hand, with a change in the oral medium, linked to the diabetic disease and to its treatment, and also to the deficiency of the immune response, which result in an imbalance of the bacterial flora, often to the benefit of pathogenic strains, and the growth of opportunistic microorganisms.

It has thus been shown that bacteria that are pathogenic for periodontal tissue, such as Porphyromonas gingivalis, Capnocytophaga ochracea, Fusobacterium nucleatum and Prevotella intermedia, are more frequently found in the oral flora of diabetics (Sbordone L. et al, (1998) Periodontal status and subgingival microbiota of insulin-dependent juvenile diabetics: a 3-year longitudinal study, J. Periodontol. 69, 120-128).

Reduction of the salival flow also promotes the occurrence of caries. The prevalence of infections mediated by Helicobacter pylori, the bacterium responsible for gastrointestinal ulcers, which is present in dental plaque and is transmitted orally, is also higher in the case of diabetics (Quadri R. et al, (2000) Helicobacter pylori infection in type 2 diabetic patients, Nutr. Metab. Cardiovasc. Dis. 10, 263-266), for whom the eradication of the bacterium is also more difficult than for normal individuals (Gasbarrini A. et al, (1999) Insulin-dependent diabetes mellitus affects eradication rate of Helicobacter pylori infection, Eur. J. Gastroenterol. Hepatol. 11, 713-716).

Numerous compositions for oral use have already been described. For example, documents WO 00/28977 and WO 99/00106 describe compositions containing at least two antioxidants as active principles, intended for use by smokers or for general use.

Thus, document WO 00/28977 describes a dental gel or paste composition comprising as active ingredients “reduced” glutathione (GSH) and a source of selenium, optionally combined with other constituents, for preventing and treating gingival symptoms.

Document Wo 99/00106 describes compositions for oral hygiene for use by smokers, comprising “reduced” glutathione and a source of selenium, and also other constituents.

However, the incorporation of GSH in significant amount into a toothpaste or a dental gel is difficult to implement industrially, both on account of the instability of this compound at room temperature and on account of its cost.

A composition for oral hygiene that is more particularly capable of preventing and/or combating oral pathologies in diabetics does not otherwise exist at the present time.

The invention is directed toward overcoming these drawbacks, by proposing a composition for oral hygiene that is suitable for diabetics and that has biological activity in a normal situation, with a product that is stable and simple to formulate.

A first subject of the invention thus relates to a composition for oral hygiene, comprising as active principles at least one fluoride-ion vector and at least one stable antioxidant, said antioxidant not being glutathione.

A second subject of the invention relates to the use of a fluoride-ion vector and an antioxidant for the preparation of a composition for oral hygiene to combat and/or prevent oral pathologies in diabetics.

According to a first aspect, the invention relates to a composition for oral hygiene, to combat and/or prevent oral pathologies in diabetics, comprising as active principles at least one fluoride-ion vector and at least one stable antioxidant, said antioxidant not being glutathione.

According to the invention, the composition comprises as first active principle at least one antioxidant, with the exception of glutathione.

According to the invention, the term “antioxidant” means a natural or unnatural compound capable of reducing the damage caused by oxygen free radicals.

Antioxidants reduce the oxidative stress of cells and exert a protective role on biological systems with respect to pathologies associated with oxygen free radicals.

Antioxidants also play a role in protection against contamination with Helicobacter pylori, the prevalence of the infection appearing to be linked to a weakness in the antioxidant defenses.

Antioxidants that may be used according to the invention are especially:

α-tocopherol and its derivatives, especially vitamin E, and tocopheryl salts;

coenzyme Q10 and its derivatives, particularly ubiquinol 10;

vanadium and vanadium salts;

vitamin C and ascorbic acid salts;

pyruvate;

nicotinic acid;

vitamin A, retinol, α-carotene, β-carotene, lycopene, lutein, zeaxanthin and other carotenoids;

thiamine, riboflavin, pyridoxine and vitamins B1, B2 and B6;

melatonin;

flavonoids;

lipoic acid;

sulfur-containing amino acids such as methionine, cysteine and N-acetylcysteine, and taurine.

Antioxidants that are preferred according to the invention are especially α-tocopherol and its derivatives, especially vitamin E, optionally in salt form, for example in acetate form.

The compositions according to the invention comprise antioxidants in an amount such that the total antioxidant content is greater than 0.0001% by weight and preferably between 0.0005% and 2% by weight relative to the total weight of said composition.

According to the invention, the composition comprises as second active principle at least one fluoride-ion vector.

According to the invention, the term “fluoride-ion vector” means a mineral or organic fluorine compound, which allows the release of fluoride ions into an aqueous medium, such as saliva.

Fluoride ions are known to increase the resistance of teeth to the acids produced from the sugars present in the oral cavity and to prevent the risk of dental caries. At high content, they also have antibacterial activity on the bacteria of dental plaque and on Helicobacter pylori.

Fluoride-ion vectors that may be used according to the invention are especially:

amine fluorides, ammonium fluoride, alkali metal fluorides, for example sodium fluoride and potassium fluoride, alkaline-earth metal fluorides, for example calcium fluoride, titanium fluoride, zinc fluoride, tin fluoride and aluminum fluoride, and also derivatives of these fluorides;

alkali metal fluorophosphates, for example potassium or sodium fluorophosphates, especially sodium monofluorophosphate, alkaline-earth metal fluorophosphates, especially calcium fluorophosphates, aluminum fluorophosphates, especially aluminum monofluorophosphate, and aluminum difluorophosphate;

sodium fluorozirconate;

fluorosilicates such as sodium fluorosilicates, for instance sodium hexafluorosilicate.

Preferred fluoride-ion vectors are especially sodium fluoride, sodium monofluorophosphate or a mixture of sodium fluoride and sodium monofluorophosphate.

The composition according to the invention comprises fluoride-ion vectors in an amount such that its fluoride-ion content is greater than 0.0001% by weight and preferably between 0.001% and 2% by weight relative to the total weight of the composition.

According to one embodiment of the invention, the composition of the invention comprises a first and a second antioxidant and at least one fluoride-ion vector.

According to this embodiment, the first antioxidant is as defined in the text hereinabove and the second antioxidant is a selenium product.

Selenium products that may be used include compounds in salt form, especially a selenate, for example sodium selenate; a selenite, for example sodium selenite; an organoselenium compound, for example a compound with an amino acid, such as selenomethionine or selenocysteine.

As antioxidants often act in synergy, the combination of two or more antioxidants therefore presents an additional advantage.

Preferred combinations of antioxidants are especially a mixture of α-tocopherol or of derivatives thereof and a sodium selenate or selenite.

It goes without saying that, when the composition of the invention comprises only one antioxidant, this antioxidant may be chosen from the selenium compounds described above.

The active principles described in the text hereinabove and the processes for preparing them are all known or commercially available.

The compositions of the invention may be in any form that is suitable for local oral application. Such a form for oral application may consist especially of a dentifrice, a spray, a chewing gum, a pastille to be sucked, a dental gel for topical application, an oral implant such as an oral patch, especially a mucoadhesive patch, or a mouthwash.

The compositions in dentifrice form may be in paste, liquid, gel, powder or mousse form.

These forms per se and the preparation thereof are well known to those skilled in the art. In addition to the fluoride-ion vectors and the antioxidants, the forms for oral application mentioned above may comprise excipients or ingredients that are conventional for each of these forms.

For example, the forms for oral application may contain anionic, amphoteric, zwitterionic, cationic or nonionic surfactants. They may also comprise thickeners, cohesion agents, sweeteners, humectants, refreshing agents, preserving agents, colorants, bleaching agents, aromatic agents or taste enhancers, essential oils of plants, plasticizers, peptizers, anti-tartar agents, agents for inhibiting the production of volatile sulfur compounds, such as zinc salts and complexes, cicatrizing agents, anti-bleeding agents, polishing agents, agents for combating dental plaque, such as chlorhexidine, hexetidine, cetylpyridinium chloride, triclosan and/or enzymes, for instance dextranase, mutanase, lysozymes, lactoferrin or peroxidases.

According to another aspect, the invention relates to the use of at least one antioxidant and of at least one fluoride-ion vector for the preparation of a composition for oral hygiene, to prevent and/or combat oral pathologies in diabetics.

According to the invention, the antioxidants and fluoride-ion vectors, and the respective concentrations thereof, are as described in the text hereinabove.

Additional characteristics and advantages of the invention will become apparent in the examples that follow.

In order to study the efficacy of an oral hygiene product of the invention intended for diabetics, compositions in dentifrice form in accordance with the invention comprising the compounds hereinbelow (expressed as weight percentages relative to the total weight of the composition) are prepared:

Composition 1

| | |
|---|---|
| sodium fluoride | 0.33 |
| sodium monofluorophosphate | 0.76 |
| DL-α-tocopherol (vitamin E) | 0.5 |
| silicas | 21.00 |
| sorbitol | 25.00 |
| sodium benzoate | 4.00 |
| phosphates | 0.3 |
| carrageenate | 1.00 |
| titanium dioxide | 1.00 |
| surfactant | 2.00 |
| preserving agent | 0.1 |
| flavoring | 1.0 |
| purified water | qs |

Composition 2

| | |
|---|---|
| sodium fluoride | 0.55 |
| DL-α-tocopherol (vitamin E) | 0.5 |
| silicas | 20.00 |
| glycerol | 32.00 |
| carboxymethylcellulose | 1.00 |
| titanium dioxide | 1.00 |
| surfactant | 1.00 |
| preserving agent | 0.30 |
| flavoring | 0.2 |
| purified water | qs |

The following results were obtained.

### EXAMPLE 1

Study of the Prolonged Presence of Vitamin E in Saliva after Brushing

The availability and remanence of vitamin E in saliva after brushing was studied in nine individuals, who used composition 1 twice a day for two days, for brushings lasting three minutes. Vitamin E was undetectable in the saliva before brushing, it reached an average concentration of 259 μg/g at the end of brushing and was still present, at an average concentration of 3 μg/g, three hours after brushing using the composition of the invention.

### EXAMPLE 2

Study of the Antibacterial Effect on the Bacteria of Dental Plaque

For comparative purposes, a dentifrice not in accordance with the invention was prepared, comprising the same compounds as in composition 1 above, with the exception of the sodium fluoride, sodium monofluorophosphate and vitamin E (placebo).

Culture media were inoculated with bacterial strains representative of strains present in dental plaque, under conditions favorable for their growth. The media containing the bacteria were then placed in contact with either composition 1 or the placebo composition, after dilution of the compositions to ½, ¼ and ⅛.

The results obtained after five minutes of contact are given in Table I below.

TABLE I

| | ½ | | ¼ | | ⅛ | |
|---|---|---|---|---|---|---|
| Dilution Strain | Comp. 1 | Placebo | Comp. 1 | Placebo | Comp. 1 | Placebo |
| Streptococcus mutans | 4.7 (6.8) | 19 (11) | 5.9 (0.6) | 27 (1.5) | 8.0 (0.4) | 25 (1.9) |
| Streptococcus salivarius | 4.7 (1.2) | 23 (3.5) | 8.1 (0.4) | 20 (3) | 12 (3.4) | 23 (2.5) |
| Lactobacillus | 5.9 (0.2) | 18 (5) | 6.5 (2.4) | 19 (2.6) | 13 (1.2) | 22 (4.5) |
| Casei | | | | | | |
| Actinomyces | 5.1 (2.3) | 20 (1.5) | 7.2 (2.5) | 22 (2.5) | 20 (1) | 29 (0.8) |
| Odontolyticus | | | | | | |
| Actinobacillus | 7.3 (0.4) | 9.0 (1.5) | 7.2 (1.0) | 9.1 (1.1) | 17 (1.0) | 17 (2.0) |
| Actinomycetemcomitans | | | | | | |
| Eikenella | 8.2 (1.5) | 11 (1.4) | 6.9 (1.1) | 11 (1.9) | 9 (0.9) | 17 (0.6) |
| Corrodens | | | | | | |
| Capnocytophaga | 3.8 (1.5) | 8.0 (0.4) | 6.2 (1.1) | 10 (0.3) | 12 (0.8) | 19 (0.8) |
| ochracea | | | | | | |
| Porphyromonas | 0.9 (0.1) | 2.0 (1.4) | 1.0 (0.1) | 2.2 (2.5) | 1.4 (1.1) | 2.3 (1.1) |
| gingivalis | | | | | | |
| Prevotella intermedia | 0.9 (0.5) | 2.2 (1.1) | 0.9 (0.4) | 2.2 (3.5) | 1.5 (5.4) | 1.9 (2.1) |
| Bacteroides | 10 (1.2) | 26 (9) | 9.8 (0.5) | 25 (20.4) | 22 (2.5) | 25 (0.6) |
| Loeschii | | | | | | |
| Prevotella | 5.0 (0.7) | 18 (3.6) | 6.7 (16.0) | 21 (3.0) | 13.5 (4.5) | 22 (1.5) |
| Melaninogenicus | | | | | | |
| Fusobacterium | 2.1 (0.1) | 16 (7.6) | 2.4 (6.8) | 15 (3.6) | 14 (3.6) | 16 (1.5) |
| nucleatum | | | | | | |
| Campylobacter | 10 (1.5) | 16 (5.0) | 9.0 (0.3) | 15 (0.3) | 17 (0.3) | 18 (10.0) |
| Rectus | | | | | | |
| Peptostreptococcus | 4.0 (0.8) | 14 (1.5) | 8.3 (0.3) | 19 (7.6) | 19 (1.5) | 22 (3.0) |
| micros | | | | | | |

The values indicated in Table I represent the number of colony-forming units (CFU), to be multiplied by 10⁵. Each of these values represents an average of three tests. The standard deviation is indicated in parentheses.

These results show that the composition of the invention reduces the number of CFUs of most of these strains, after a contact time of only five minutes.

### EXAMPLE 3

Antibacterial Effect on Helicobacter pylori

For comparative purposes, a dentifrice not in accordance with the invention (placebo), comprising the compounds indicated for composition 1 above, with the exception of sodium fluoride, sodium monofluorophosphate and vitamin E (placebo), was prepared. A culture medium was inoculated with a strain of Helicobacter pylori, under conditions favorable for its growth. The media containing the bacteria were then placed in contact with either composition 1 of the invention or the placebo composition, after dilution of the compositions to ½, ¼, ⅛ and 1/16.

The results obtained after five minutes, one hour and twenty-four hours of contact are indicated in Tables II (placebo) and III (composition 1) below.

TABLE II

| Dilution | | | | |
|---|---|---|---|---|
| Contact | | | | |
| time | 1/2 | 1/4 | 1/8 | 1/16 |
| 5 minutes | 10 (2) | 20 (2.1) | 35 (1.7) | 35 (3.4) |
| 1 hour | 9.5 (1.2) | 17 (1.9) | 35 (0.8) | 44 (2.8) |
| 24 hours | 9.2 (0.2) | 9.5 (0.3) | 42 (0.5) | 48 (20) |

TABLE III

| Dilution | | | | |
|---|---|---|---|---|
| Contact | | | | |
| time | 1/2 | 1/4 | 1/8 | 1/16 |
| 5 minutes | 5 (0.2) | 8 (0.5) | 11 (0.8) | 25 (2.5) |
| 1 hour | 2.9 (0.1) | 5.2 (0.3) | 10 (1.9) | 19 (5.6) |
| 24 hours | 1.2 (0.3) | 1.4 (0.4) | 10 (0.9) | 40 (4.2) |

The values indicated in Tables II and III represent the number of colony-forming units (CFU), to be multiplied by 10⁵ Each of these values represents an average of three tests. The standard deviation is indicated in parentheses.

These results show that the composition of the invention greatly reduces the number of Helicobacter pylori CFUs, after a contact time of only five minutes.

### EXAMPLE 4

Improvement of the Condition of the Gums

For comparative purposes, two compositions not in accordance with the invention were prepared:

one comprising the compounds indicated in composition 1 above, with the exception of sodium fluoride, sodium monofluorophosphate and vitamin E (placebo),

the other comprising the compounds indicated in composition 1 above, with the exception of vitamine E (fluorinated control).

The efficacy of composition 1 was demonstrated in a clinical study performed on twenty-four volunteers. During a first phase of fourteen days, all the individuals brushed their teeth with the placebo. Next, they used, twice a day for twenty-one days, the dentifrice allocated to them, from the dentifrice of the invention (composition 1) and the two other dentifrices not in accordance with the invention, mentioned above, under the following conditions: a portion of the dentition (teeth 32 to 37) was protected from the mechanical action of the brushing by a cover, into which two milliliters of the dentifrice were introduced, while the other teeth were brushed normally for two minutes.

Each individual received the three dentifrices successively for twenty-one days, in an order determined by randomization, with a period of fourteen days under placebo between each treatment.

The state of inflammation of the gums in the portion of the mouth not brushed during the treatment was evaluated, on replicas, by measuring the gingival thickness at the start of the gum attached from the start of the tooth, according to the three-dimensional method described by Jovanovski V. et al, (2000) Analysis of the morphology of oral structures from 3-D co-ordinate data, Monogr. Oral Sci. 17, 73-129.

The results obtained after twenty-one days of treatment are indicated in Table IV below.

TABLE IV

| | | Fluorinated | |
|---|---|---|---|
| Treatment | Composition 1 | control | Placebo |
| Thickness of the | −82.6 (68.6)** | −19.8 (76.4) | −15.4 (111.7) |
| gum | | | |
| (difference in | | | |
| μm) | | | |

The values indicated in Table IV represent the difference in the thickness (in μm) of the gum between the start and the end of the treatment. Each of these values represents an average on 20 to 22 individuals. The standard deviation is indicated in parentheses.

The statistical analysis (Duncan's Multiple Range) demonstrates a greater reduction in the thickness of the gum after use of composition 1 according to the invention (**, p<0.01) than after use of the fluorinated control or of the placebo. Given the dimensions of the gum at the point of measurement, this effect represents a reduction of more than 10% in the thickness of the gum.

These results show that the composition of the invention, due to the presence of an antioxidant that is stable in the formulation and in sufficient amount, significantly improves the condition of the gums.

Other examples of compositions in the form of chewing gums or mouthwashes are given below.

Chewing Gum

The following composition (in weight percentages relative to the total weight of the composition) was used to prepare a chewing gum in accordance with the invention:

| | |
|---|---|
| sodium fluoride | 0.005 |
| DL-α-tocopherol (vitamin E) | 0.005 |
| taurine | 2.0 |
| gum | 32.0 |
| xylitol | 35.0 |
| glycerol | 2.0 |
| flavoring | 4.0 |
| sorbitol | qs |

Mouthwash

The following composition (in weight percentages relative to the total weight of the composition) was used to prepare a mouthwash in accordance with the invention:

| | |
|---|---|
| sodium fluoride | 0.01 |
| sodium selenate | 0.0005 |
| glycerol | 5.00 |
| preserving agent | 0.20 |
| xylitol | 7.00 |
| surfactant | 1.00 |
| colorant | 0.20 |
| flavoring | 0.05 |
| purified water | qs |

1. A composition for oral hygiene, to combat and/or prevent oral pathologies in diabetics, containing as active principles vitamin E and at least one fluoride-ion vector, in a fluoride-ion content of greater than 0.0001% by weight relative to the total weight of the composition.

2. The composition as claimed in claim 1, characterized in that wherein the vitamin E is present in salt form.

3. The composition as claimed in claim 1 wherein the fluoride-ion vector is amine fluoride, ammonium fluoride, alkali metal fluorides, alkaline-earth metal fluorides, and also derivatives of these fluorides; aluminum fluorophosphates, alkali metal fluorophosphates, alkaline-earth metal fluorophosphates, for example calcium aluminum difluorophosphate; sodium fluorozirconate; or fluorosilicates.

4. The composition as claimed in claim 3 wherein the fluoride-ion vector is sodium fluoride, sodium monofluorophosphate or a mixture thereof.

5. The composition as claimed in claim 1 wherein it comprises as active principles a second antioxidant.

6. The composition as claimed in claim 5, wherein the second antioxidant is a selenium product.

7. The composition as claimed in claim 6, wherein the selenium product is a selenate, a selenite, or an organoselenium compound.

8. The composition as claimed in, claim 1 wherein the total antioxidant content is greater than 0.0001% by weight relative to the total weight of said composition.

9. The composition as claimed in claim 1 which may be used in the form of a dentifrice wherein it comprises the following compounds, expressed as weight percentages relative to the total weight of the composition:

| | |
|---|---|
| sodium fluoride | 0.33 |
| sodium monofluorophosphate | 0.76 |
| DL-α-tocopherol (vitamin E) | 0.5 |
| silicas | 21.00 |
| sorbitol | 25.00 |
| sodium benzoate | 4.00 |
| phosphates | 0.3 |
| carrageenate | 1.00 |
| titanium dioxide | 1.00 |
| surfactant | 2.00 |
| preserving agent | 0.1 |
| flavoring | 1.0 |
| purified water | qs |

10. The composition as claimed in claim 1 which may be used in the form of a dentifrice, wherein it comprises the following compounds, expressed as weight percentages relative to the total weight of the composition:

| | |
|---|---|
| sodium fluoride | 0.55 |
| DL-α-tocopherol (vitamin E) | 0.5 |
| silicas | 20.00 |
| glycerol | 32.00 |
| carboxymethylcellulose | 1.00 |
| titanium dioxide | 1.00 |
| surfactant | 1.00 |
| preserving agent | 0.30 |
| flavoring | 0.2 |
| purified water | qs |

11. The composition as claimed in claim 1 wherein it is formulated in the form of a dentifrice, a spray for oral application, a chewing gum, a pastille to be sucked, an oral patch or a mouthwash.

12. A method to prevent and/or combat oral pathologies in diabetics which comprises administering to a patient in need thereof a composition according to claim 1.

13. A method as claimed in claim 12, wherein the antioxidant is α-tocopherol, vitamin E, tocopheryl salts, vanadium, vanadium salts, coenzyme Q10 and its derivatives, vitamin C, ascorbic acid salts, pyruvate, nicotinic acid, vitamin A, retinol, α-carotene, β-carotene, lycopene, lutein, zeaxanthin and other carotenoids, thiamine, riboflavin, pyridoxine, and vitamins B1, B2 and B6, melatonin, flavonoids, lipoic acid, or sulfur-containing amino acids.

14. A method as claimed in claim 12, wherein the fluoride-ion vector is especially amine fluoride, ammonium fluoride, alkali metal fluorides, alkaline-earth metal fluorides, and also derivatives of these fluorides; aluminum fluorophosphates, alkali metal fluorophosphates, alkaline-earth metal fluorophosphates, aluminum difluorophosphate; sodium fluorozirconate; or fluorosilicates.

15. A method as claimed in claim 12 wherein it comprises as active principles a first and a second antioxidant and at least one fluoride-ion vector.

16. A method as claimed in claim 15, wherein the second antioxidant is a selenium product.

17. A composition according to claim 1 wherein the fluoride-ion content is between 0.001% and 2% by weight.

18. A composition according to claim 2 wherein the vitamin E is present in acetate salt form.

19. A composition according to claim 3 wherein the alkali metal fluoride is sodium fluoride and potassium fluoride; the alkaline-earth metal fluorides is calcium fluoride, titanium fluoride, zinc fluoride, tin fluoride or aluminum fluoride, or derivatives of these fluorides; the alkali metal fluorophosphates is sodium and potassium fluorophosphates; the alkaline-earth metal fluorophosphates is calcium fluorophosphates, or aluminum fluorophosphates; and the fluorosilicates is sodium fluorosilicates.

20. A composition according to claim 19 wherein the sodium fluorophosphates is sodium monofluorophosphate; the aluminum fluorophosphates is aluminum monofluorophosphate, and the sodium fluorosilicates is sodium hexafluorosilicate.

21. A composition according to claim 7 wherein the selenate is sodium selenate; the selenite is sodium selenite, and the organselenium compound is selenomethionine or selenocysteine.

22. A composition according to claim 8 wherein the total antioxidant content is between 0.0005% and 2% by weight.

23. A method according to claim 13 wherein the coenzyme Q10 and its derivatives is ubiquinol 10; and sulfur-containing amino acids are methionine, cysteine, N-acetylcysteine, or taurine.

24. A method according to claim 14 wherein the alkali metal fluorides is sodium fluoride or potassium fluoride; the alkaline-earth metal fluorides is calcium fluoride, titanium fluoride, zinc fluoride, tin fluoride, aluminum fluoride, and derivatives of these fluorides; the alkali metal fluorophosphates is sodium or potassium fluorophosphates; the alkaline-earth metal fluorophosphates is calcium fluorophosphates or aluminum fluorophosphates; and the fluorosilicates is sodium fluorosilicates.

25. A method according to claim 24 wherein the sodium fluorophosphates is sodium monofluorophosphate; the aluminum fluorophosphates is aluminum monofluorophosphate; and the sodium fluorosilicates is sodium hexafluorosilicate.

26. A method according to claim 16 wherein the selenium product is a selenate, selenite, or an organoselenium compound.

27. A method according to claim 26 wherein the selenate is sodium selenate; the selenite is sodium selenite; and the organoselenium compound is selenomethionine or selenocysteine.
